(19) United States
(12) Patent Application Publication
(10) Pub. No.: US 2026/0306992 A1
(43) Pub. Date: Oct. 8, 2026

(54) METHOD FOR TREATING A FLUID, FLUID DISCHARGE DEVICE FOR A FLUID AND ASSOCIATED SYSTEM

(71) Applicant: Langenstein & Schemann GmbH, Coburg (DE)

(72) Inventors: Friedrich HERDAN, Coburg (DE); Lothar BAUERSACHS, Weidhausen (DE); Harald BARNICKEL, Coburg (DE)

(21) Appl. No.: 19/638,419

(22) Filed: Apr. 3, 2026

(30) Foreign Application Priority Data
Apr. 3, 2025 (DE) 102025113095.7

(51) Int. Cl. B01D 53/04; B01D 53/053; C07C 7/12

(52) U.S. Cl. CPC B01D 53/0446; B01D 53/053; C07C 7/12; B01D 2253/102; B01D 2256/245; B01D 2257/504; B01D 2258/05; B01D 2259/40032

(57) ABSTRACT

The invention relates to a method for treating a fluid or fluid mixture, in particular a biogas mixture, a fluid discharge device for a fluid or fluid mixture, in particular for a biogas mixture stored in a fluid adsorption storage and a system comprising a fluid discharge device and a first fluid adsorption storage.

The invention relates to a method, in particular for treating a fluid or a fluid mixture, in particular a biogas mixture, a fluid discharge device for a fluid or fluid mixture, in particular for a biogas mixture stored in a fluid adsorption storage, and a system comprising a fluid discharge device and a first fluid adsorption storage.

Biogas is understood above and below to mean a gas produced by the fermentation of biomass, for example in a biogas plant such as part of a landfill site, a sewage treatment plant or an agricultural business. Biogas can be used, for example, to supply local heating systems, power stations or combustion engines in vehicles. However, biogas has an average calorific value of only 60% compared to normal pipeline-quality natural gas. To produce high-quality, pipeline-grade biomethane, biogas is collected in pipelines and pumped to a biomethane processing plant (also known as an RNG plant), where it is processed in several steps in large purification plants. The most cost-intensive step is the separation of CH₄ and CO₂.

However, many agricultural biogas producers are located in remote areas where there are no pipelines to a biomethane processing plant. For this reason, biogas producers are dependent on processes that allow biogas to be transported to a biomethane processing plant.

One way of transporting biogas is to fill mobile tanks, for example in the form of a tank in a tanker vehicle or a tank trailer for a motor vehicle, with biogas and then transport them to a biomethane processing plant. Such a mobile tank can be a fluid adsorption storage, for example.

In fluid adsorption storages, fluids, in particular biogas, are adsorbed, for example, onto carbon-based adsorption materials, known as adsorbents (singular: adsorbent). Such adsorption materials or adsorbents include, for example, activated carbon, periodically mesoporous organosilicon dioxide, controllably porous glasses, mesoporous silicon dioxide, carbon-mesoporous Korea, monolithic carbon, in particular monolithic nanoporous carbon, or monomodal microporous carbon-based adsorbents.

Adsorption is understood above and below to mean the attachment of gases, molecules or dissolved substances to the interface of a solid or liquid phase, the adsorption material or the adsorbent. A molecule of a fluid that is not adsorbed by the adsorption material but remains in the fluid bulk phase is referred to as an adsorptive. The molecules of the fluid that are bound to the adsorbents, i.e., the adsorption material, are referred to as adsorbates. The adsorbent and the adsorbate are collectively referred to as the adsorbent-adsorbate complex.

The removal of fluids stored in fluid adsorption storages is performed at least partially by desorption. This is the reverse reaction of adsorption. During desorption, the gas adsorbed to the adsorption material, the adsorbed molecules or the adsorbed dissolved substances are detached from the adsorption material by applying energy.

Adsorption materials are often very porous, which means they have a high specific surface area. This is often accompanied by limited thermal conductivity. The adsorption of a molecule is typically an exothermic process in which adsorption enthalpy is released. In other words, the adsorption material is usually heated by adsorption. However, heating the adsorption material can promote desorption in the adsorption material. In other words, heating the adsorption material can reduce the adsorption capacity of the adsorption material. The same applies to desorption. The desorption of a molecule is typically an endothermic process in which desorption enthalpy is extracted from the adsorption material. In other words, the adsorption material is usually cooled by desorption. However, cooling the adsorption material can counteract desorption in the adsorption material. In other words, cooling the adsorption material can increase the adsorption capacity of the adsorption material.

Adsorption and desorption processes are important for the process of storing fluids in adsorption storage systems and for the process of extracting fluids from adsorption storage systems. For rapid emptying of the adsorption storage system, a low adsorption capacity or adsorptivity of the adsorbents for the corresponding adsorbates can be advantageous at the moment of extracting fluids from adsorption storage systems, whereas for rapid filling of the adsorption storage at the moment of storing fluids in adsorption storages, a high adsorption capacity or adsorptivity of the adsorbents for the corresponding adsorbates may be advantageous. In addition to the adsorptivity, in particular the selective adsorptivity for the respective adsorbates, the adsorption and desorption processes are generally also dependent on the system temperature and the partial pressure or concentration of the adsorptive or adsorptives.

As already explained above, previous adsorption and desorption processes can influence the temperature that prevails in the adsorption material of a fluid adsorption storage during subsequent adsorption and desorption processes. In addition, the partial pressures in fluid adsorption storages change during the filling or emptying of the fluid adsorption storages. The maximum volume of fluid that can be extracted from a fluid adsorption storage in a given period of time, or the maximum volume of fluid that can be filled into a fluid adsorption storage in a given period of time, depends not only on the temperature and pressure conditions at the start of these respective processes, but also on the change in temperature and pressure conditions over the course of the extraction or filling process, i.e., on the temperature gradient over time and the pressure gradient over time.

In known methods for storing and/or extracting fluids in or from fluid adsorption storages, the complex interactions between adsorption and desorption processes, temperature and pressure changes and the mass flow of the fluid are usually not taken into account or are not taken into account sufficiently, with the result that the adsorption capacity of the adsorbents is higher than desired during withdrawal processes from fluid adsorption storage on the one hand and lower than desired during filling processes of fluid adsorption storage on the other. With regard to extraction processes from fluid adsorption storage systems, this can have the disadvantage that a fluid adsorption storage system can only be emptied slowly or incompletely. Incomplete emptying of a fluid adsorption storage can in turn be associated with the disadvantage that the fluid adsorption storage can store a smaller additional amount of fluid in a subsequent filling process. In other words, the storage capacity of fluid adsorption storages is not optimally utilised if the complex interactions between adsorption and desorption processes, temperature and pressure changes, and the mass flow of the fluid are not sufficiently taken into account.

The invention is therefore based on the task of specifying a new method, a new device and a new system which at least largely avoids the disadvantages of the prior art.

This task is solved by a method with the features of claim 1. Furthermore, this task is solved by a fluid discharge device with the features of claim 29 and by a system with the features of claim 34. Advantageous embodiments and further developments are specified in the dependent claims.

The method according to the invention is a method, in particular for treating a fluid or fluid mixture, in particular a biogas mixture, comprising:
(a) providing a first fluid adsorption storage, which
(a1) comprises at least one fluid with molecules of at least two different chemical substances, in particular a biogas mixture with methane molecules and carbon dioxide molecules, and
(a2) comprises at least one first adsorbent (or: adsorption material) to which at least some of the molecules of the fluid are adsorbed as adsorbates, while some of the molecules are not adsorbed as adsorptives (or free molecules), and wherein the adsorptivities of the molecules of the different chemical substances are different, in particular the methane has a lower adsorptivity with the adsorbent, which preferably comprises activated carbon, than the carbon dioxide,
(a3) has at least one outlet valve, and
(a4) is under a storage pressure from a high-pressure range, in particular between 2 and 10 MPa,
(b) providing a fluid discharge device comprising at least one supply line, at least one discharge line and at least one suction pump,
(c) wherein, in a first separation mode, the following process steps (d), (e) and (f) with the sub-steps (f1) to (f3) are performed on or with the first fluid adsorption storage:
(d) connecting the supply line of the fluid discharge device to the closed outlet valve of the first fluid adsorption storage;
(e) opening the outlet valve of the first fluid adsorption storage and allowing the fluid to flow out of the first fluid adsorption storage through the opened outlet valve, in a first operating mode, in particular in a passive mode without suction pressure from the suction pump, until the storage pressure reaches a low-pressure range, in particular below 2 MPa, and/or as long as the outflowing fluid predominantly contains molecules of the chemical substance with the lower adsorptivity,
(f) following the first operating mode, in particular following the passive mode, operating the fluid discharge device in a second operating mode, in particular in an active mode, with the following features:
(f1) generation of a suction pressure at the open outlet valve of the first fluid adsorption storage by means of the at least one suction pump;
(f2) desorbing or detaching adsorbates and/or molecules of the chemical substance with the higher adsorptivity that are still attached after the first operating mode, in particular after the passive mode, from the at least one adsorbent in the first fluid adsorption storage by means of the suction pressure of the suction pump, and
(f3) removing these desorbed or detached adsorbates and adsorptives from the first fluid adsorption storage by means of the fluid discharge device, whereby the removed fluid now predominantly contains molecules of the chemical substance with the higher adsorptivity.

A suction pump is understood above and below to be a pump which can suck in a medium to be conveyed, for example a fluid, by generating a negative pressure. A suction pump can also be referred to as a vacuum pump. The suction pump can be designed, for example, as a reciprocating piston pump, rotary vane pump, slide valve pump, rolling piston pump or screw vacuum pump.

The first fluid adsorption storage may, for example, comprise adsorbents with a gravimetric methane storage capacity of at least 0.13 kg of methane per 1 kg of adsorbents. Furthermore, the first fluid adsorption storage may, for example, have a volumetric storage capacity of at least 0.08 kg of methane per 1 litre of fluid adsorption storage volume at a temperature of 20° C. and a pressure of 6 MPa.

Connecting the supply line of the fluid discharge device to the outlet valve, opening the outlet valve, and operating the suction pump can be performed and monitored by an operator. Alternatively, these process steps can also be performed in an automated form by a suitable system.

The first fluid adsorption storage can, for example, be a tanker vehicle tank which, after being filled with biogas at a biogas production site, is driven to a biomethane processing plant and emptied there into a storage of the biomethane processing plant for further processing.

The fluid discharge device can, for example, be located at the storage facility of the biomethane processing plant, at the first fluid adsorption storage or at the tanker vehicle.

Advantageously, the suction pump for generating the suction pressure is operated at least when the pressure in the fluid adsorption storage has dropped from essentially 6 MPa to essentially 2.5 MPa after the outlet valve has been opened.

The invention has the advantage that the suction pressure generated by the suction pump removes more adsorbates from the one or more first adsorbents, allowing a larger quantity of fluid or a larger volume of fluid to be sucked out of the fluid adsorption storage in a mass flow through the fluid discharge device. On the one hand, this is advantageous because the amount of fluid or volume of fluid that can be removed from a fluid adsorption storage per storage filling is increased by removing the adsorbates from the surfaces of the adsorbents. On the other hand, this has the advantage that the adsorbents in the fluid adsorption storage are freed from as many adsorbates as possible, thus increasing the free surface area of the adsorbents available for subsequent adsorption processes, for example in a subsequent filling process of the fluid adsorption storage. In other words, the adsorbents of the fluid adsorption storage are optimised in an advantageous manner for a subsequent filling process with a fluid such as biogas by the method according to the invention. This can also be referred to as conditioning of the adsorbents located in the fluid adsorption storage.

Previously and subsequently, a first operating mode can be understood to mean, on the one hand, a passive mode without suction pressure from the suction pump and, on the other hand, a mode in which the suction pump generates a first suction pressure or a suction pressure along a first pressure curve. Furthermore, a second operating mode can be understood to mean, on the one hand, an active mode with suction pressure from the suction pump and, on the other hand, a mode in which the suction pump generates a second suction pressure that is higher than the first suction pressure or a suction pressure along a second pressure curve. For example, the suction pump is operated in the first operating mode with a suction pressure of 100 kPa and in the second operating mode with a suction pressure of 1 MPa. Alternatively, in the first operating mode, the suction pump is initially operated at a suction pressure of 100 kPa, with the suction pressure rising linearly or non-linearly to a suction pressure of 500 kPa over the duration of the first operating mode, and wherein the suction pump is initially operated in the second operating mode with a suction pressure of 1 MPa and the suction pressure increases linearly or non-linearly to a suction pressure of 3 MPa over the duration of the second operating mode.

According to one embodiment of the method according to the invention, the method further comprises the following steps:
providing a primary fluid storage; and
connecting the discharge line of the fluid discharge device to at least one valve of the primary fluid storage;
wherein, in the second operating mode, in particular in the active mode, the fluid is fed through the discharge line of the fluid discharge device into the primary fluid storage.

In an exemplary embodiment, the primary fluid storage device is part of a biomethane processing plant to which the primary fluid adsorption storage device was transported by means of a tanker vehicle. For example, in a first step, a fluid adsorption storage device filled with biogas is transported from a production site, such as an agricultural business, to a biomethane processing plant. The biogas stored in this exemplary fluid adsorption storage has, for example, a methane concentration of 60 vol. % and a carbon dioxide concentration of 40 vol. %. The pressure in the filled fluid adsorption storage is, for example, 6 MPa. If an outlet valve of this fluid adsorption storage is opened and biogas escapes through this outlet valve under a high pressure gradient, for example into a methane storage, the biogas escaping under the high pressure gradient typically has a methane concentration that is at least 10 vol. % higher than the methane concentration present in the fluid adsorption storage before the outlet valve was opened. For example, the methane concentration of the escaping biogas shortly after the outlet valve is opened is 75 vol. %. However, the local pressure gradient between the fluid adsorption storage and the methane storage decreases as the fluid adsorption storage is emptied. Typically, the biogas escaping under a low pressure gradient has a carbon dioxide concentration that is higher than the methane concentration present in the fluid adsorption storage before the outlet valve is opened. In other words, after the outlet valve is opened, biogas with a higher methane concentration escapes initially, with the methane concentration decreasing as the fluid adsorption storage is emptied and the carbon dioxide concentration of the escaping gas increasing compared to the concentrations in the fluid adsorption storage before the outlet valve is opened. For example, the methane concentration of the escaping biogas may be 75 vol. % and the carbon dioxide concentration of the escaping biogas may be 25 vol. % if the pressure difference between the fluid adsorption storage and the methane storage is 6 MPa shortly after the outlet valve is opened, whereas the methane concentration of the escaping biogas can be 20 vol. % and the carbon dioxide concentration of the escaping biogas can be 80 vol. % if the pressure difference between the fluid adsorption storage and the methane storage is only 2.5 MPa after a certain time after opening the outlet valve. This relationship between the pressure conditions present when emptying a fluid adsorption storage on the one hand and the gas concentrations of the biogas on the other hand can be exploited for gas separation purposes.

In the aforementioned exemplary embodiment, for example, the fluid adsorption storage transported to the biomethane processing plant can first be emptied into a methane storage under a high pressure gradient. As soon as the pressure difference between the fluid adsorption storage and the methane storage has changed by a certain amount and, as a result, biogas with a lower methane concentration flows into the methane storage, the fluid adsorption storage and the methane storage can be separated fluidically. The biogas remaining in the fluid adsorption storage at a lower pressure can then be advantageously discharged into a primary fluid storage. In the aforementioned exemplary embodiment, for example, biogas with a carbon dioxide concentration of 80 vol. % flows into the primary fluid storage after the fluid adsorption storage has first been partially emptied into a methane storage under a high pressure gradient. In this example, carbon dioxide is therefore primarily discharged into the primary fluid storage, whereas methane was primarily discharged into the methane storage. This can be associated with the advantage that a two-stage emptying of the fluid adsorption storage enables simple and cost-effective separation of methane and carbon dioxide.

According to a further embodiment of the method according to the invention, the primary fluid storage is a second fluid adsorption storage comprising at least a second adsorbent to which molecules of the fluid or adsorbates of the different chemical substances attach or are adsorbed.

The second adsorbents may be of the same type as the first adsorbents or may differ from the type of the first adsorbents. In an exemplary embodiment, biogas is discharged into the second fluid adsorption storage. In this exemplary embodiment, the second adsorbents of the second fluid adsorption storage advantageously have an adsorption capacity that is optimised, i.e., increased, with respect to the fluid that is present in a lower concentration in the fluid escaping after the outlet valve is opened. For example, the second adsorbents adsorb the methane escaping from the first fluid adsorption storage at a low concentration under a low pressure gradient better than the escaping carbon dioxide. In other words, in this embodiment, due to the optimised adsorption capacity of the adsorbents, the methane molecules carried in the fluid tend to attach themselves to the second adsorbents of the second fluid adsorption storage more strongly than carbon dioxide molecules. Optimization of the adsorption capacity with respect to a specific fluid can be achieved, for example, by selecting the material of the adsorbents, by a certain surface structuring of the adsorbents or by adjusting the pore size and pore distribution in the adsorbents.

According to a further embodiment of the method according to the invention, the method further comprises attaching one or more second adsorbates from the fluid phase of the at least one fluid to the one or more second adsorbents of the second fluid adsorption storage.

According to a further embodiment of the method according to the invention, a second separation mode is performed with the second fluid adsorption storage, which comprises the corresponding features of the first separation mode according to feature (c) for the second fluid adsorption storage instead of the first fluid adsorption storage. In other words, in this embodiment, the following process steps are performed on or with the second fluid adsorption storage in the second separation mode:
connecting the supply line of the fluid discharge device to the closed outlet valve of the second fluid adsorption storage;
opening the outlet valve of the second fluid adsorption storage and allowing the fluid to flow out of the second fluid adsorption storage through the opened outlet valve, in particular in a first operating mode, in particular a passive mode without suction pressure from the suction pump, until the storage pressure of the second fluid adsorption storage reaches a low-pressure range, in particular below 2 MPa, and/or as long as the fluid flowing out of the second fluid adsorption storage predominantly contains molecules of the chemical substance with the lower adsorptivity,
following the first operating mode, in particular following the passive mode, operating the fluid discharge device in the second operating mode, in particular in the active mode, with the following features:
generation of a suction pressure at the open outlet valve of the second fluid adsorption storage by means of the at least one suction pump;
desorbing or detaching adsorbates and/or molecules of the chemical substance with the higher adsorptivity that are still attached after the first operating mode, in particular after the passive mode, from the at least one adsorbent in the second fluid adsorption storage by means of the suction pressure of the suction pump, and
removing these desorbed or detached adsorbates and adsorptives from the second fluid adsorption storage by means of the fluid discharge device, whereby the removed fluid now predominantly contains molecules of the chemical substance with the higher adsorptivity.

According to a further embodiment of the method according to the invention, the method further comprises:
providing a secondary fluid storage device; and
connecting the discharge line of the fluid discharge device to the secondary fluid storage;
wherein, in the first separation mode, the following process steps are further performed on or with the first fluid adsorption storage:
   opening the outlet valve of the first fluid adsorption storage and allowing the fluid to flow out of the first fluid adsorption storage through the opened outlet valve in the first operating mode, in particular in the passive mode without suction pressure from the suction pump, until the storage pressure reaches a low-pressure range, in particular below 2 MPa, and/or as long as the outflowing fluid predominantly contains molecules of the chemical substance with the lower adsorptivity;
   flowing of the fluid in the first operating mode, in particular in the passive mode, out of the first fluid adsorption storage through the discharge line of the fluid discharge device into the secondary fluid storage.

In an exemplary embodiment, the fluid is biogas, the secondary fluid storage is a methane storage that primarily stores methane, and the primary fluid storage is a carbon dioxide storage that primarily stores carbon dioxide. In this example, in the first operating mode of the first separation mode, in particular in the passive mode of the first separation mode, biogas is first discharged into the methane storage under a high pressure gradient, whereby the biogas discharged in this process has a high methane concentration. In the second operating mode of the first separation mode, which follows the first operating mode of the first separation mode, in particular in the active mode of the first separation mode following the passive mode of the first separation mode, biogas is discharged into the carbon dioxide storage under a lower pressure gradient in this example, whereby the biogas discharged in this way has a high carbon dioxide concentration. A high methane concentration in the first operating mode of the first separation mode is to be understood as a higher methane concentration than in the second operating mode of the first separation mode. In particular, a high methane concentration in the passive mode of the first separation mode is to be understood as a higher methane concentration than in the active mode of the first separation mode. A high carbon dioxide concentration in the second operating mode of the first separation mode is understood to mean a higher carbon dioxide concentration than in the first operating mode of the first separation mode. In particular, a high carbon dioxide concentration in the active mode of the first separation mode is understood to mean a higher carbon dioxide concentration than in the passive mode of the first separation mode.

Connecting the discharge line of the fluid discharge device to the secondary fluid storage is advantageously performed before connecting the discharge line of the fluid discharge device to the primary fluid storage. Alternatively, the fluid discharge device may have a changeover module that can fluidically connect the discharge line of the fluid discharge device either to the secondary fluid storage or to the primary fluid storage. In this case, the discharge line of the fluid discharge device can be connected to both the secondary fluid storage and the primary fluid storage essentially simultaneously.

According to a further embodiment of the method according to the invention, the fluid, which in the second operating mode of the first separation mode, in particular in the active mode of the first separation mode, is directed through the discharge line of the fluid discharge device into the second fluid adsorption storage, is stored in the second fluid adsorption storage at a storage pressure from a high-pressure range, in particular between 2 and 10 MPa, particularly using a compressor. A compressor used for high-pressure storage of the fluid in the second fluid adsorption storage may, for example, be arranged in the fluid discharge device. Alternatively, a separate compressor may be used for high-pressure storage of the fluid in the second fluid adsorption storage.

According to a further embodiment of the method according to the invention, the method further comprises:
providing a pure fluid storage device;
connecting the discharge line of the fluid discharge device to the pure fluid storage;
wherein, in the second separation mode, the following process steps are further performed on or with the second fluid adsorption storage:
   opening the outlet valve of the second fluid adsorption storage and allowing fluid to flow out of the second fluid adsorption storage through the opened outlet valve in a first operating mode of the second separation mode, in particular in a passive mode of the second separation mode without suction pressure from the suction pump, until the storage pressure in the second fluid adsorption storage reaches a low-pressure range, in particular below 2 MPa, and/or as long as the outflowing fluid predominantly contains molecules of the chemical substance with the lower adsorptivity,
   following the first operating mode of the second separation mode, in particular following the passive mode of the second separation mode, operating the fluid discharge device in a second operating mode of the second separation mode, in particular in an active mode of the second separation mode, with the following features:
      generating a suction pressure at the open outlet valve of the second fluid adsorption storage by means of the at least one suction pump;
      desorbing or detaching adsorbates and/or molecules of the chemical substance with the higher adsorptivity that are still attached after the first operating mode of the second separation mode, in particular after the passive mode of the second separation mode, from the second adsorbent in the second fluid adsorption storage by means of the suction pressure of the suction pump, and
      removing these desorbed or detached adsorbates and adsorptives from the second fluid adsorption storage via the fluid discharge device, whereby the removed fluid now predominantly contains molecules of the chemical substance with the higher adsorptivity
      flowing the desorbed or detached adsorbates and adsorptives out of the second fluid adsorption storage through the discharge line of the fluid discharge device into the pure fluid storage.

In an exemplary embodiment, the second fluid adsorption storage is, for example, a first carbon dioxide storage device and the pure fluid storage device is a second carbon dioxide storage device. The fluid is, for example, biogas. By discharging biogas into the first carbon dioxide storage under a low pressure gradient, the biogas stored in the first carbon dioxide storage as a second fluid adsorption storage in a high carbon dioxide concentration, for example a carbon dioxide concentration of essentially 80 vol. %. Preferably, the carbon dioxide-rich biogas discharged in the second operating mode of the first separation mode, in particular in the active mode of the first separation mode, is compressed by means of a compressor and thus stored in the first carbon dioxide storage as a second fluid adsorption storage in such a way that it is present in the first carbon dioxide storage under high-pressure, approximately 6 MPa. In the exemplary embodiment discussed here, the fluid discharge device comprises the aforementioned compressor for compressing the carbon dioxide-rich biogas in the first discharge step. Subsequently, the carbon dioxide-rich biogas under a pressure of 6 MPa in the carbon dioxide storage is partially discharged into a methane storage in a first operating mode of a second separation mode, in particular in a passive mode of the second separation mode. In the first operating mode of the second separation mode, in particular in the passive mode of the second separation mode, the biogas is discharged into the methane storage at a high flow rate or mass flow due to the high pressure gradient between the first carbon dioxide storage and the methane storage. In this rapid discharge, methane is primarily discharged with the discharged biogas, while carbon dioxide initially remains attached to the adsorbents of the first carbon dioxide storage. In other words, the biogas discharged in the first operating mode of the second separation mode, in particular in the passive mode of the second separation mode, initially has a high methane concentration, whereby the methane concentration of the discharged biogas decreases as the first carbon dioxide storage is progressively discharged and as the pressure gradient between the first carbon dioxide storage and the methane storage decreases until the carbon dioxide concentration of the discharged biogas is higher than the methane concentration of the discharged biogas. In this exemplary embodiment, the first operating mode of the second separation mode, in particular the passive mode of the second separation mode, ends and a subsequent second operating mode of the second separation mode, in particular an active mode of the second separation mode, begins when the carbon dioxide concentration of the discharged biogas is greater than the methane concentration of the biogas discharged from the first carbon dioxide storage. If the biogas stored in the first carbon dioxide storage as a second fluid adsorption storage is now discharged in the second operating mode of the second separation mode, in particular in the active mode of the second separation mode, under a low pressure gradient into a second carbon dioxide storage as a pure fluid storage, the discharged biogas again has a higher carbon dioxide concentration than the biogas stored in the first carbon dioxide storage before the first operating mode of the second separation mode, in particular before the passive mode of the second separation mode. For example, the biogas discharged from the first carbon dioxide storage in the second operating mode of the second separation mode, in particular in the active mode of the second separation mode, has a carbon dioxide concentration of essentially 99 vol. %. In accordance with the two successive separation modes according to this exemplary embodiment, a concentration of a specific fluid, such as carbon dioxide gas, can be achieved by cascading the first operating mode and the second operating mode, in particular by cascading the passive mode and the active mode, taking into account the prevailing pressure conditions. For example, in the second carbon dioxide storage as a pure fluid storage after the second operating mode of the second separation mode, in particular after the active mode of the second separation mode, essentially pure carbon dioxide is present according to the aforementioned exemplary embodiment.

According to a further embodiment of the method according to the invention, the method further comprises the following steps:
providing a mass flow controller, and
controlling the mass flow through the fluid discharge device to a mass flow target value, wherein the mass flow target value is in particular time-dependent.

Typically, a mass flow controller comprises a sensor for measuring a mass flow flowing through the mass flow controller. The measured value of this mass flow is compared with the mass flow target value. In order to obtain a mass flow that corresponds to the mass flow target value, a control valve in the mass flow controller typically opens or reduces the size of the effective flow opening through the mass flow controller.

It has been found that faster emptying of the fluid adsorption storage cools the fluid adsorption storage, i.e., the adsorbents in the fluid adsorption storage. In other words, a large mass flow or a high flow rate through the mass flow controller during the removal of fluid from the fluid adsorption storage is associated with a greater temperature drop, particularly at the surface of the adsorbents. However, a greater temperature drop at the surface of the adsorbents generally leads to an increase in the adsorption capacity of the adsorbents. An increased adsorption capacity of the adsorbents is usually undesirable during emptying of the fluid adsorption storage.

A reduction in the mass flow or flow rate during emptying of the fluid adsorption storage by means of the mass flow controller can counteract cooling of the adsorbents and, as a result, also increased adsorption capacity of the adsorbents during emptying of the fluid adsorption storage. In other words, the use of a mass flow controller that regulates the mass flow through the fluid discharge device to a mass flow target value can achieve that more adsorbate molecules can be removed from warmer surfaces of the adsorbents and thus a larger total amount of material or a larger total volume of a fluid stored in the fluid adsorption storage can be removed from the fluid adsorption storage. A warmer surface of the adsorbents or a larger total amount of a fluid is understood here to mean a warmer surface of the adsorbents or a larger total amount of the fluid compared to the surface temperature of the adsorbents or compared to the total amount of fluid that can be removed when the fluid-adsorption storage without using a mass flow controller that reduces the mass flow or flow rate. The possibility of removing a larger total amount of substance from the fluid or a larger total volume of fluid from the fluid adsorption storage in a discharge or removal process can also be described as an improvement in removal efficiency or discharge efficiency.

It should also be noted that a change in the mass flow or flow rate can have different effects on the adsorption capacity of the adsorbents with respect to the respective adsorptive, depending on the adsorptive. For example, the adsorption capacity of a certain type of adsorbent changes more strongly towards carbon dioxide than towards methane as a result of cooling due to a large mass flow or a high flow rate of biogas from a fluid adsorption storage. For example, in a rapid discharge process of a fluid adsorption storage system, the tendency of carbon dioxide to accumulate on the surface of the adsorbents increases more than the tendency of methane to accumulate on the surface of the adsorbents as a result of the cooling of the adsorbents. In other words, cooling the adsorbents can, for example, increase the adsorption tendency towards carbon dioxide more than towards methane. This relationship can be advantageously exploited for the purpose of separating carbon dioxide and methane.

In an exemplary embodiment, the mass flow controller regulates the mass flow through the fluid discharge device in the second discharge step to a higher mass flow target value than in the first discharge step. For example, the first fluid adsorption storage is rapidly discharged in the second discharge step, causing the adsorbents to cool significantly and, as a result, the adsorption capacity for carbon dioxide to increase more than that for methane. The biogas discharged in a rapid partial discharge therefore has a higher methane concentration compared to a slow discharge. Accordingly, methane can be discharged primarily from the first fluid adsorption storage by means of a rapid partial discharge, while carbon dioxide tends to adsorb to the adsorbents as a result of the cooling of the adsorbents. The rapid discharge step for discharging primarily methane can advantageously be followed by a slow discharge step for discharging primarily carbon dioxide. In such a slow discharge step, the mass flow controller can, for example, regulate the mass flow or flow rate through the mass flow controller to a lower target value compared to the mass flow or flow rate during rapid discharge. The slow discharge of the first fluid adsorption storage in the subsequent removal step results, for example, in the temperature in the adsorbents in the first fluid adsorption storage cooling down less and thus the adsorption capacity of the adsorbents with respect to carbon dioxide being increased less. Consequently, in a slow discharge of the first fluid adsorption storage, more carbon dioxide can be released from the warmer surfaces of the adsorbents, or in this way, carbon dioxide can be released from the surfaces of the adsorbents by a lower suction pressure. Consequently, a larger total amount of material or a larger total volume of carbon dioxide can be removed from the first fluid adsorption storage by means of a slow discharge of the first fluid adsorption storage. In other words, slow discharge of the first fluid adsorption storage increases the discharge efficiency with regard to carbon dioxide. This effect can be referred to as the first effect. The biogas discharged in a slow discharge has a higher carbon dioxide concentration compared to a fast discharge.

In the aforementioned exemplary embodiment, methane was primarily discharged from the storage in a preceding rapid partial discharge process of the first fluid adsorption storage. At the time of a subsequent slow discharge process, the fluid concentrations of the biogas present in the first fluid adsorption storage have consequently changed in favor of carbon dioxide, i.e., after the rapid discharge process, the carbon dioxide concentration in the first fluid adsorption storage is higher than before the rapid discharge process. In the subsequent slow discharge process, the discharged biogas therefore has an increased carbon dioxide concentration due to the preceding rapid discharge process. This effect can be referred to as the second effect.

In the case where, as in the aforementioned exemplary embodiment, a rapid discharge process of the fluid adsorption storage is followed by a slow discharge process, the increased carbon dioxide concentration of the biogas discharged in the slow discharge process is typically caused by an interaction of the two effects mentioned above, i.e., the first and second effects.

In an exemplary embodiment, the mass flow controller regulates the mass flow or flow rate flowing through the mass flow controller as a function of time. For example, the mass flow or flow rate is regulated by the mass flow controller to a higher value over a first period of time after an outlet valve of the first fluid adsorption storage is opened by means of the mass flow controller, and to a lower value over a second period of time following the first period of time.

According to a further embodiment of the method according to the invention, the level of the suction pressure is time-dependent and the suction pressure is generated by the suction pump along a predetermined or predeterminable time curve.

For example, shortly after opening the outlet valve of the first fluid adsorption storage, a lower suction pressure can initially be generated and, after a certain discharge time, a higher suction pressure can be generated. In an exemplary embodiment, shortly after opening an outlet valve of a first fluid adsorption storage filled with biogas, when the pressure gradient between the interior of the first fluid adsorption storage and a methane storage to be filled is, for example, 6 MPa, no suction pressure is generated by a suction pump. Only after a certain period of time after the outlet valve has been opened, when a certain drop in the pressure gradient between the interior of the first fluid adsorption storage and the progressively filling methane storage has occurred, does the suction pump generate a suction pressure in order to continue to remove a high quantity of material or a high volume of fluid from the first fluid adsorption storage. Increasing the suction pressure over time after opening the outlet valve can have the advantage of increasing the total amount of material that can be removed from the first fluid adsorption storage or the total volume of fluid that can be removed from it. In other words, generating the suction pressure over time along a predetermined or predeterminable time curve can increase the effective storage capacity of the first fluid adsorption storage.

In an exemplary embodiment, the suction pump generates no suction pressure in a first time period, low suction pressure in a second time period after the first time period, and higher suction pressure in a third time period after the second time period. Alternatively, the suction pump can generate a suction pressure that, for example, increases continuously after the outlet valve of a first fluid adsorption storage is opened.

In a further embodiment of the method according to the invention, the method further comprises providing a control unit that controls the operation of the suction pump and/or the mass flow controller.

The control unit may, for example, be part of the fluid discharge device. Advantageously, the control unit controls the operation of the suction pump and/or the mass flow controller in a semi-automated or fully automated manner. For example, the control unit is designed or configured to optimize the speed of discharge of the first fluid adsorption storage by controlling the mass flow controller and the suction pump in such a way that the discharge efficiency for a first fluid, such as carbon dioxide, is increased in a first discharge step, and the discharge efficiency for a second fluid, such as methane, is increased in a second discharge step. For example, the control unit controls the operation of the mass flow controller shortly after opening an outlet valve of a first fluid adsorption storage so that the flow rate through the mass flow controller is high, so that methane is primarily discharged from the first fluid adsorption storage, whereas after a certain period of time after opening the outlet valve, the control unit controls the operation of the suction pump so that the suction pressure generated by the suction pump counteracts the pressure gradient decreasing due to the progressive discharge. In this way, firstly, separation of methane and carbon dioxide can be achieved and, secondly, a higher effective storage capacity of the first fluid adsorption storage can be achieved.

According to a further embodiment of the method according to the invention, the method further comprises the following steps:
providing a measuring unit for detecting measured values of one or more of the following measured variables:
   pressure within the supply line of the fluid discharge device,
   temperature within the supply line of the fluid discharge device,
   mass flow through the fluid discharge device; and
   fluid concentration within the supply line of the fluid discharge device; and
detecting measured values of the one or more measured variables by the measuring unit.

According to a further embodiment of the method according to the invention, the control unit receives a control signal corresponding to the one or more measured values detected, and the control unit activates the suction pump when the level of the detected pressure corresponding to the received control signal falls below a first threshold value.

According to a further embodiment of the method according to the invention, the first threshold value is generally in a range between 250 kPa and 4 MPa, preferably in a range between 2 MPa and 3 MPa, and particularly preferably in a range between 2.3 MPa and 2.7 MPa, or the first threshold value is generally in a range between two and a half times and forty times, preferably between twenty and thirty times, particularly preferably between twenty-three and twenty-seven times atmospheric pressure outside the first fluid adsorption storage.

According to a further embodiment of the method according to the invention, the suction pump generates the suction pressure level as a function of the received control signal, in particular as a function of the level of the detected pressure corresponding to the received control signal.

In an exemplary embodiment, the suction pump generates, for example, a first lower suction pressure when a measuring unit within a supply line of the fluid discharge device detects a temperature below, for example, 0° C., the control unit then generates a control signal in response to this detected temperature, and the suction pump receives this control signal. Furthermore, according to this exemplary embodiment, the suction pump generates a second higher suction pressure when the measuring unit detects a temperature above 0° C. within the supply line of the fluid discharge device, the control unit then generates a control signal in response to this detected temperature, and the suction pump receives this control signal.

In a further exemplary embodiment, the suction pump generates, for example, a first lower suction pressure when a measuring unit detects a methane concentration above 20 vol. % in the biogas discharged from a first fluid adsorption storage, the control unit then generates a control signal in response to this detected methane concentration, and the suction pump receives this control signal. Furthermore, according to this exemplary embodiment, the suction pump generates a second, higher suction pressure when the measuring unit detects a methane concentration below 20 vol. % in the biogas discharged from the first fluid adsorption storage, the control unit then generates a control signal in response to this detected methane concentration, and the suction pump receives this control signal.

In a particularly advantageous exemplary embodiment, the suction pump generates, for example, a first lower suction pressure when a measuring unit within the supply line of a fluid discharge device detects a pressure above 2.5 MPa during the discharge of biogas, the control unit then generates a control signal in response to this detected pressure, and the suction pump receives this control signal. Furthermore, according to this particularly advantageous exemplary embodiment, the suction pump generates a second higher suction pressure when the measuring unit within the supply line of the fluid discharge device detects a pressure below 2.5 MPa during the removal of biogas, the control unit then generates a control signal in response to this detected pressure, and the suction pump receives this control signal.

According to a further embodiment of the method according to the invention, the suction pump generates the suction pressure level depending on the detected pressure level corresponding to the received control signal, wherein the suction pump is operated in a first operating mode if the level of the detected pressure corresponding to the received control signal is above a second threshold value, and the suction pump is operated in a second operating mode if the level of the detected pressure corresponding to the received control signal is below the second threshold value, wherein the pressure generated in the second operating mode is greater than the pressure generated in the first operating mode, and wherein the second threshold value is generally between 100 kPa and 1.1 MPa, preferably in a range between 150 kPa and 750 kPa, and particularly preferably in a range between 200 kPa and 300 kPa, or wherein the second threshold value is generally in a range between one and eleven times, preferably between one and a half and seven and a half times, particularly preferably between two and three times, the atmospheric pressure outside the first fluid adsorption storage.

According to a further embodiment of the method according to the invention, the at least one fluid is a fluid mixture comprising a first fluid in a first fluid concentration and a second fluid in a second fluid concentration.

According to a further embodiment of the method according to the invention, the first fluid concentration in the fluid mixture flowing through the discharge line of the fluid discharge device during the third discharge is greater than the first fluid concentration in the fluid mixture flowing through the discharge line of the fluid discharge device during the first discharge.

For example, biogas stored in a first fluid adsorption storage is discharged in a slow discharge process into a first carbon dioxide storage, which is a second fluid adsorption storage. The biogas discharged into the first carbon dioxide storage has, for example, a carbon dioxide concentration of 80 vol. %. The biogas stored in the first carbon dioxide storage after completion of the slow discharge process, which primarily comprises carbon dioxide, is discharged, for example, into a second carbon dioxide storage in a second slow discharge process. The carbon dioxide concentration of the biogas discharged in the second slow discharge process is, for example, 99 vol. %. In the second carbon dioxide storage, almost exclusively carbon dioxide is stored according to this exemplary embodiment. The second carbon dioxide storage can thus be referred to as a pure fluid storage. A cascade of two slow discharge processes, as in this exemplary embodiment, can serve to concentrate a certain fluid in the fluid mixture, such as carbon dioxide.

According to a further embodiment of the method according to the invention, the first fluid concentration in the fluid mixture flowing through the discharge line of the fluid discharge device during the third discharge is essentially 99 vol. %.

According to a further embodiment of the method according to the invention, the operation of the suction pump and/or the mass flow controller during the first discharge is controlled by the control unit such that that the first fluid concentration in the fluid mixture flowing through the discharge line of the fluid discharge device during the first discharge is greater than the first fluid concentration in the fluid mixture stored in the fluid adsorption storage prior to the first discharge.

According to a further embodiment of the method according to the invention, the temporal temperature gradient and/or the temporal pressure gradient within the supply line of the fluid discharge device is controlled by means of the operation of the suction pump and/or the mass flow controller during the first discharge such that that during the first discharge from the one or more first adsorbents, more adsorbate molecules of the first fluid are removed and transferred to the fluid phase than adsorbate molecules of the second fluid.

For example, during a first discharge of biogas from a first fluid adsorption storage, a mass flow controller reduces the mass flow such that the temperature within the supply line of the fluid discharge device changes more slowly compared to a first discharge without restriction of the mass flow. As a result of the reduced temperature drop in the adsorbents of the first fluid adsorption storage, the adsorption capacity of the adsorbents with respect to a first fluid, such as carbon dioxide, increases less sharply compared to a first discharge without restriction of the mass flow. Due to the reduced adsorption capacity of the adsorbents with respect to carbon dioxide, more carbon dioxide molecules than methane molecules are removed from the adsorbents in this exemplary embodiment. In this respect, more carbon dioxide molecules than methane molecules pass into the fluid phase during the first discharge in this exemplary embodiment.

According to a further embodiment of the method according to the invention, the first fluid is carbon dioxide and the second fluid is methane.

According to a further embodiment of the method according to the invention, the method for conditioning the one or more adsorbents is performed before refilling the first fluid adsorption storage. Furthermore, according to this embodiment of the invention, the removal of the one or more first adsorbates from the one or more first adsorbents of the first fluid adsorption storage increases the surface area of the one or more first adsorbents available for the adsorption of adsorbates.

For example, the fluid discharge device removes a larger total amount of material, such as carbon dioxide, from the surface of the adsorbents of the first fluid adsorption storage than is possible with known methods, and this material is removed by the suction pressure. Accordingly, a larger free surface area of the adsorbents is available for filling processes following the emptying of the first fluid adsorption storage, for example with biogas. This can have the advantage that the effective storage capacity of the first fluid adsorption storage is increased using the method according to the invention.

According to a further embodiment of the method according to the invention, the fluid discharge device comprises a changeover valve which, in a first position, fluidically connects the outlet valve of the first fluid adsorption storage to the secondary fluid storage and, in a second position, fluidically connects the outlet valve of the first fluid adsorption storage to the primary fluid storage, wherein the changeover valve is designed or configured to receive a changeover signal from the control unit for switching from the first position to the second position.

In an exemplary embodiment, the changeover valve is switched to the first position before methane is primarily discharged from the first fluid adsorption storage to the secondary fluid storage in a rapid discharge process of a first fluid adsorption storage. According to this exemplary embodiment, the changeover valve is further switched to the second position before carbon dioxide is primarily discharged from the first fluid adsorption storage into the primary fluid storage device in a slow discharge process of the first fluid adsorption storage.

According to a further embodiment of the method according to the invention, the changeover signal is generated by the control unit as a function of the measured values of the one or more measured variables detected by the measuring unit.

In an exemplary embodiment, a changeover signal that switches the changeover valve from the first position to the second position is generated, for example, when a fluid concentration value detected by the measuring unit, such as a methane concentration, falls below a threshold value of 50 vol. % within the supply line of the fluid discharge device. This can have the advantage that the first fluid adsorption storage is automatically connected to a methane storage unit as long as methane is primarily discharged from the first fluid adsorption storage, and is automatically connected to a carbon dioxide storage unit as long as carbon dioxide is primarily discharged from the first fluid adsorption storage.

According to a further embodiment of the method according to the invention, the first discharge, the second discharge and the third discharge are controlled automatically by the control unit.

According to a further embodiment of the method according to the invention, the operation of the suction pump and/or the mass flow controller during the second discharge preceding the first discharge is controlled by the control unit such that that the second fluid concentration in the fluid mixture flowing through the discharge line of the fluid discharge device during the second discharge of the fluid mixture is greater than the second fluid concentration in the fluid mixture stored in the fluid adsorption storage prior to the second discharge.

In an exemplary embodiment, the mass flow controller is operated during a rapid discharge preceding a slow discharge of the first fluid adsorption storage in such a way that there is a high flow rate through the mass flow controller during the rapid discharge. In particular, due to the strong cooling of the adsorbents, methane is primarily discharged from the first fluid adsorption storage during the rapid discharge process. According to the aforementioned exemplary embodiment, the methane concentration of the discharged biogas is, for example, 70 vol. %, whereas the methane concentration of the biogas stored in the first fluid adsorption storage prior to discharge was 60 vol. %.

According to a further embodiment of the method according to the invention, the temporal temperature gradient and/or the temporal pressure gradient within the supply line of the fluid discharge device is controlled by means of the operation of the suction pump and/or the mass flow controller during the second discharge, that during the second discharge, more adsorbate molecules of the second fluid are removed from the one or more first adsorbents and pass into the fluid phase than adsorbate molecules of the first fluid.

According to a further embodiment of the method according to the invention, the temporal pressure gradient within the supply line of the fluid discharge device during the second removal is greater than during the first removal.

The fluid discharge device according to the invention is a device, in particular for use in one of the aforementioned methods, for treating a fluid, in particular a biogas mixture stored in a fluid adsorption storage, wherein the device comprises a suction pump, and wherein the device is designed or suitable for generating, by means of the suction pump, a suction pressure which detaches one or more first adsorbates, in particular methane molecules, methanol molecules, hydrogen molecules or carbon dioxide molecules, from the first adsorbent and to remove them in fluid phase from the first fluid adsorption storage via the fluid discharge.

According to one embodiment of the device according to the invention, the level of the suction pressure is time-dependent and the suction pressure is generated by the suction pump along a predetermined or predeterminable time curve.

According to a further embodiment of the device according to the invention, the device further comprises a mass flow controller which is designed or configured to control the mass flow through the fluid discharge device to a mass flow target value, wherein the mass flow target value is in particular time-dependent.

According to a further embodiment of the device according to the invention, the fluid discharge device further comprises a control unit which is designed or configured to control the operation of the suction pump and/or the mass flow controller.

According to a further embodiment of the device according to the invention, the fluid discharge device further comprises a measuring unit for detecting measured values of one or more of the following measured variables:
pressure within the supply line of the fluid discharge device,
temperature within the supply line of the fluid discharge device,
mass flow through the fluid discharge device; and
fluid concentration within the supply line of the fluid discharge device.

The system according to the invention is a system particularly for use in one of the methods according to the invention, comprising a fluid discharge device according to the invention and a first fluid adsorption storage comprising at least one first adsorbent.

According to one embodiment of the system according to the invention, the at least one first adsorbent is selected from a group comprising activated carbon, periodically mesoporous organosilicon dioxide, controllably porous glasses, mesoporous silicon dioxide, carbon-mesoporous Korea, monolithic carbon, in particular monolithic nanoporous carbon, and monomodal microporous carbon-based adsorbents.

According to a further embodiment of the system according to the invention, the first fluid adsorption storage comprises an outlet valve.

According to a further embodiment of the system according to the invention, the first fluid adsorption storage can be fluidically connected to the supply line of the fluid discharge device via the outlet valve.

According to a further embodiment of the system according to the invention, the system further comprises a primary fluid storage device.

According to a further embodiment of the system according to the invention, the primary fluid storage device is a second fluid adsorption storage comprising at least one second adsorbent selected from a group comprising activated carbon, periodically mesoporous organosilicon dioxide, controllably porous glasses, mesoporous silicon dioxide, carbon-mesoporous Korea, monolithic carbon, in particular monolithic nanoporous carbon, and monomodal microporous carbon-based adsorbents.

According to a further embodiment of the system according to the invention, the primary fluid storage comprises an inlet valve.

According to a further embodiment of the system according to the invention, the primary fluid storage can be fluidically connected to the discharge line of the fluid discharge device via the inlet valve.

According to a further embodiment of the system according to the invention, the first fluid adsorption storage is arranged in or on a motor vehicle, or the first fluid adsorption storage is arranged in or on a trailer of a motor vehicle.

According to a further embodiment of the system according to the invention, the fluid discharge device is arranged in or on the motor vehicle according to the preceding embodiment, or the fluid discharge device is arranged in or on a trailer of the motor vehicle according to the preceding embodiment.

According to a further embodiment of the system according to the invention, the primary fluid storage is arranged in or on the motor vehicle according to one of the two preceding embodiments, or the primary fluid storage is arranged in or on a trailer of the motor vehicle according to one of the two preceding embodiments.

According to a further embodiment of the system according to the invention, the motor vehicle is a tanker vehicle according to one of the three preceding embodiments.

The invention is further explained below with reference to exemplary embodiments. Reference is also made to the drawings, which schematically show:

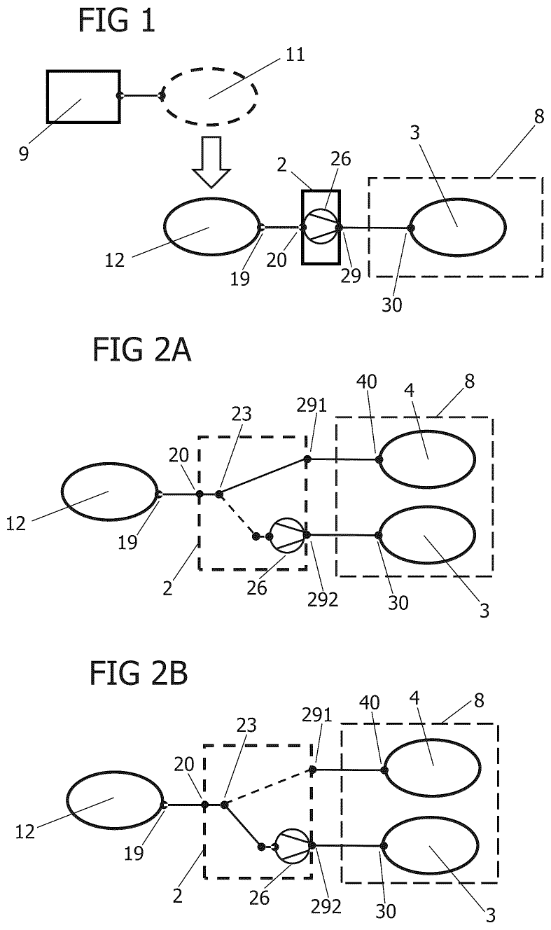

FIG. 1 a system comprising a first fluid adsorption storage, a fluid discharge device and a primary fluid storage,

FIG. 2A a system comprising a first fluid adsorption storage, a primary fluid storage device, a secondary fluid storage device and a fluid discharge device in a passive mode,

FIG. 2B a system comprising a first fluid adsorption storage, a primary fluid storage device, a secondary fluid storage device and a fluid discharge device in an active mode,

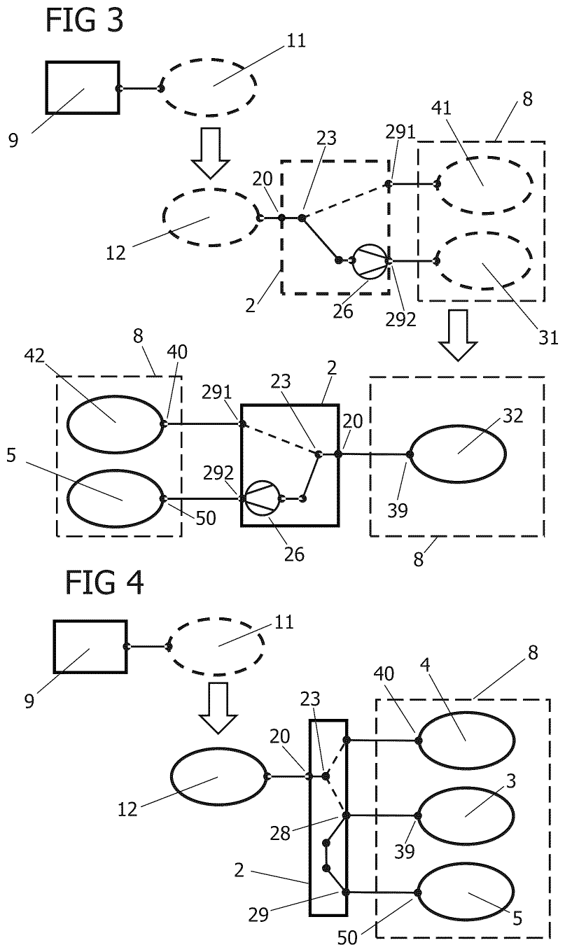

FIG. 3 a system comprising a primary fluid storage, a secondary fluid storage, a pure fluid storage and a fluid discharge device in a first and second separation mode,

FIG. 4 a system comprising a first fluid adsorption storage, a fluid discharge device, a primary fluid storage device, a secondary fluid storage device and a pure fluid storage,

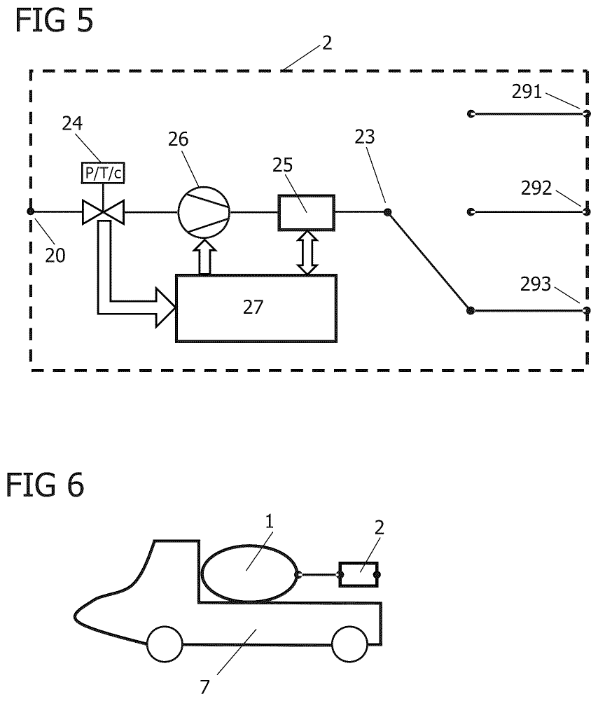

FIG. 5 a fluid discharge device for discharging a fluid in a system according to one of FIG. 1-3,

FIG. 6 a system comprising a first fluid adsorption storage, a fluid discharge device and a tanker vehicle.

Corresponding parts and sizes are designated by the same reference numerals in FIG. 1 to 6.

According to FIG. 1, a fluid adsorption storage 11 to be filled is filled at a first point in time at a biogas production plant 9 with biogas comprising methane and carbon dioxide and then transported to a biomethane processing plant 8. At a later second point in time after the earlier first point in time, the filled fluid adsorption storage 12 is fluidically connected to the primary fluid storage 3 via the fluid discharge device 2, wherein the primary fluid storage 3 is part of the biomethane processing plant 8. The filled fluid adsorption storage comprises monolithic carbon as adsorbents, i.e., as adsorption material. The fluid discharge device 2 comprises a supply line 20, a suction pump 26 and a discharge line 29. An outlet valve 19 of the fluid adsorption storage 12 is connected to the fluid discharge device 2 via the supply line 20. An inlet valve 30 of the primary fluid storage 3 is connected to the discharge line 29 of the fluid discharge device 2. The fluid discharge device 2 discharges the biogas stored in the fluid adsorption storage 12 into the primary fluid storage 3. The suction pump 26 generates a suction pressure which releases adsorbates from the adsorbents in the fluid adsorption storage, whereby the adsorbates comprise methane and carbon dioxide molecules. The suction pressure causes the dissolved methane and carbon dioxide molecules, i.e., the dissolved adsorbates, as well as other methane and carbon dioxide molecules present in the fluid bulk phase, to be discharged in fluid form from the fluid adsorption storage into the primary fluid storage 3. The biogas stored in the primary fluid storage 3 by this process can be further processed in subsequent processes in the biomethane processing plant 8.

According to FIG. 2A, a fluid discharge device 2 has a changeover valve 23 which, in a first position, connects the supply line 20 of the fluid discharge device 2 to a first discharge line 291 connected to a secondary fluid storage 4. In a second position of the changeover valve 23, the supply line 20 is connected via a suction pump 26 and a second discharge line 292 to a primary fluid storage 3. The position of the changeover valve 23 shown in FIG. 2A corresponds to the first position in which the supply line 20 is connected to the first discharge line 291. In the first position of the changeover valve 23, the fluid discharge device 2 is operated in a passive mode. Shortly after an outlet valve 19 is opened, methane gas flows primarily from a fluid adsorption storage 12 filled with biogas under high pressure. Carbon dioxide molecules are typically more strongly bound to the adsorbents present in the fluid adsorption storage than methane molecules. As a result, after the outlet valve 19 is opened, the methane concentration in the fluid adsorption storage 12 initially decreases more sharply than the carbon dioxide concentration. In other words, in the initial phase of removing biogas from the fluid adsorption storage 12, the relative proportion of carbon dioxide in the fluid adsorption storage 12 increases. In addition, desorption processes on the surface of the adsorbents typically cause the interior of the fluid adsorption storage 12 to cool down. However, cooling the surface of the adsorbents generally increases the adsorption capacity of the adsorbents for carbon dioxide more than their adsorption capacity for methane. Thus, in the first phase of removal, the tendency to retain carbon dioxide and remove primarily methane by means of the fluid discharge device 2 is further enhanced. Advantageously, the biogas discharged in the passive mode of the fluid discharge device 2, which primarily comprises methane, is discharged into the secondary fluid storage 4. The secondary fluid storage 4, which is to be filled primarily with methane in the first phase of discharge, can also be referred to as a methane storage. After a certain period of time after opening the outlet valve 19, firstly, the pressure in the fluid adsorption storage 12 has decreased and, secondly, the relative proportion of carbon dioxide in the biogas remaining in the fluid adsorption storage 12 has increased. As a result, in a second phase of discharging the biogas, carbon dioxide typically flows primarily through the outlet valve 19 into the supply line 20 of the fluid discharge device 2. In order to prevent the methane storage 4 from filling with biogas that does not primarily comprise methane during the second phase of biogas removal, the changeover valve 23 is switched to the second position after the first phase of removal, i.e., connected to the primary fluid storage 3 via the suction pump 26 and the second discharge line 292.

According to FIG. 2B, a fluid discharge device 2 has a changeover valve 23 which, in a first position, connects the supply line 20 of the fluid discharge device 2 to a first discharge line 291 connected to a secondary fluid storage 4. In a second position of the changeover valve 23, the supply line 20 is connected via a suction pump 26 and a second discharge line 292 to a primary fluid storage 3. The position of the changeover valve 23 shown in FIG. 2B corresponds to the second position in which the supply line 20 is connected to the second discharge line 292. In the second position of the changeover valve 23, the fluid discharge device 2 is operated in an active mode. After the end of a passive mode of the fluid discharge device 2, for example as shown in FIG. 2A, in a second phase of discharge, typically carbon dioxide flows through the outlet valve 19 into the supply line 20 of the fluid discharge device 2, as shown in FIG. 2B. Advantageously, the biogas discharged in the active mode of the fluid discharge device 2, which primarily comprises carbon dioxide, is discharged into the primary fluid storage 3. The primary fluid storage 3, which is to be filled primarily with carbon dioxide in the second phase of discharge, can also be referred to as a carbon dioxide reservoir. The suction pump 26 generates a suction pressure which removes adsorbate molecules from the adsorbents of the fluid adsorption storage 12 and discharges them together with adsorptives located in the fluid bulk phase from the fluid adsorption storage 12. The suction pressure generated by the suction pump 26 causes adsorbate molecules to be released from the adsorbents, particularly in the second phase of removal, when the pressure in the fluid adsorption storage 12 has already decreased. Without the additional suction pressure, these molecules would remain attached to the surface of the adsorbents. In other words, the suction pressure generated increases the total amount of material that can be removed from the fluid adsorption storage 12 or the total volume of biogas that can be removed. Furthermore, the use of the suction pump 26 frees a larger proportion of the surface of the adsorbents from adsorbate molecules, so that a larger number of adsorbate molecules can attach themselves to the adsorbents in any subsequent filling process of the fluid adsorption storage 12 emptied after the end of the passive mode. The process therefore increases the effective storage capacity of the fluid adsorption storage 12. This is advantageous for the transport of biogas between a biogas production plant 9 and a biomethane processing plant 8.

According to FIG. 3, a fluid adsorption storage 11 to be filled is filled with biogas comprising methane and carbon dioxide at a biogas production plant 9 from a first point in time and then transported to a biomethane processing plant 8. From the fluid adsorption storage 12 filled under high pressure, methane is primarily discharged into a secondary fluid storage 41 to be filled at a later second point in time after the earlier first point in time via a supply line 20, a changeover valve 23 and a first discharge line 291 in a passive mode of the fluid discharge device 2. The secondary fluid storage 41, which is to be filled primarily with methane in the passive mode of the fluid discharge device 2, can also be referred to as a methane storage. From the fluid adsorption storage 12, which is now only partially filled, carbon dioxide is primarily discharged into a primary fluid storage 31 to be filled in an active mode at a later third point in time after the earlier second point in time via the supply line 20, the changeover valve 23 and a second discharge line 292. The primary fluid storage 31, which is to be filled primarily with carbon dioxide in the active mode of the fluid discharge device 2, can also be referred to as a carbon dioxide storage.

Compared to the pressure conditions in passive mode, there is a lower pressure gradient in active mode between the fluid adsorption storage 12 and the primary fluid storage 31 to be filled. Due to this lower pressure gradient between the fluid adsorption storage 12 and the primary fluid storage unit 31 to be filled, the fluid adsorption storage 12 generally empties more slowly in active mode, i.e., at a lower flow rate or with a lower mass flow into the primary fluid storage 31 to be filled, compared to the partial emptying in passive mode. The discharge phase in active mode can therefore also be referred to as a slow discharge phase. Slow emptying in active mode can also be supported or enforced by a mass flow controller 25 of the fluid discharge device 2, which is not shown. As already explained, in active mode of the fluid discharge device 2, carbon dioxide is primarily discharged into the primary fluid storage 32. For example, the carbon dioxide concentration of the biogas discharged in active mode into the primary fluid storage 31 to be filled is essentially 80 vol. %.

The preceding discharge of primarily methane into the methane storage 41 to be filled in passive mode and the subsequent discharge of primarily carbon dioxide into the carbon dioxide storage 31 to be filled can be collectively referred to as the first separation cascade. In the first separation cascade, the fluid discharge device 2 is operated in a separation mode that comprises the passive mode and the subsequent active mode.

The primary fluid storage 31 to be filled is a fluid adsorption storage whose adsorbents comprise monolithic carbon. According to the embodiment shown in FIG. 3, the primary fluid storage 31 is filled with biogas having a carbon dioxide concentration of essentially 80 vol. % such that, after completion of the filling process, a pressure of essentially 6 MPa prevails inside the primary fluid storage 31. A compressor (not shown) can be used to compress the carbon dioxide-rich biogas in the primary fluid storage 31.

According to FIG. 3, the first separation cascade, in which biogas from the fluid adsorption storage 12 was first discharged into the methane storage 41 and then into the carbon dioxide storage 31, is followed by a second separation cascade, in which biogas from the high-pressure primary fluid storage 32 is sequentially discharged into a secondary fluid storage 42 and then into a pure fluid storage 5. The primary fluid storage 32 filled under high pressure is advantageously the carbon dioxide storage 31 filled in the first separation cascade. In the second separation cascade, at a fourth point in time after the earlier third point in time, i.e., after the first separation cascade, the biogas stored in the primary fluid storage 32 is first discharged via an inlet valve 40 into a secondary fluid storage 42 via a fluid discharge device 2, which is advantageously the same as the fluid discharge device 2 in the first separation cascade. For this purpose, the filled primary fluid storage 32 is connected to the supply line 20 of the fluid discharge device 2 via an outlet valve 39. The supply line 20 connected to the primary fluid storage 32 is connected via a changeover valve 23 in a first position to a first discharge line 291 of the fluid discharge device 2, whereby the first discharge line 291 is connected to the inlet valve 40 of the secondary fluid storage 42. The secondary fluid storage 42 may be identical to the secondary fluid storage 41, which may also be referred to as a methane reservoir. Alternatively, the secondary fluid storage 42 may be a second secondary fluid storage.

The primary fluid storage 32, filled with biogas having a carbon dioxide concentration of 80 vol. % at a pressure of 6 MPa, is partially emptied into the secondary fluid storage 42 at the fourth point in time in a passive mode of the fluid discharge device 2. The discharge phase in passive mode of the fluid discharge device 2 during the second separation cascade is another rapid discharge phase. Rapid partial emptying of the primary fluid storage 32, i.e., partial emptying at a high flow rate through the fluid discharge device 2 during this discharge phase, is caused on the one hand by the high pressure gradient between the primary fluid storage 32 and the secondary fluid storage 42. Furthermore, the rapid emptying can be further supported or forced by a mass flow controller 25, not shown in the fluid path between the changeover valve 23 and the first discharge line 291, and by a second suction pump 26, not shown in this fluid path. In the passive mode of the fluid discharge device 2, methane is primarily discharged from the primary fluid storage 32 into the secondary fluid storage 42.

In the active mode of the fluid discharge device 2, at a later fifth point in time after the earlier fourth point in time, the biogas remaining in the primary fluid storage 32 is discharged from the partially emptied primary fluid storage 32 via the fluid discharge device 2 into a pure fluid storage 5. For this purpose, the supply line 20 connected to the primary fluid storage 32 is connected via the changeover valve 23 in a second position to a second discharge line 292 of the fluid discharge device 2, wherein a suction pump 26 is connected upstream of the second discharge line 292 and the second discharge line 292 is also connected to an inlet valve 50 of the pure fluid storage 5.

After the end of passive mode and at the start of active mode of the fluid discharge device 2, the pressure in the partially filled primary fluid storage 32 is essentially 2 MPa. The discharge phase in active mode during the second separation cascade is a slow discharge phase. Slow emptying of the primary fluid storage 32, i.e., emptying at a low flow rate through the fluid discharge device 2 during this discharge phase, is caused on the one hand by the low pressure gradient between the primary fluid storage 32 and the pure fluid storage 5. On the other hand, the slow emptying can also be further supported or enforced by a mass flow controller 25, not shown in the fluid path between the changeover valve 23 and the second discharge line 292, and the suction pump 26. In the slow discharge phase in active mode during the second separation cascade, carbon dioxide is primarily discharged from the primary fluid storage 32 into the pure fluid storage. It should be noted here that the carbon dioxide concentration of the biogas discharged in the active mode of the second separation cascade is higher than the carbon dioxide concentration of the biogas discharged in the active mode of the first separation cascade. This is due in particular to the fact that the biogas stored in the primary fluid storage 32 after the first separation cascade has a higher carbon dioxide concentration than the biogas stored in the primary fluid storage 12 before the first separation cascade. Furthermore, in the passive mode of the second separation cascade, methane was primarily discharged from the primary fluid storage 32, while carbon dioxide initially remained more strongly attached to the adsorbents. The biogas removed in this embodiment during the slow removal phase in the active mode of the second separation cascade has a carbon dioxide concentration of essentially 99 vol. %.

By switching the changeover valve 23 in the fluid discharge device 2, biogas produced in the biogas production plant 9 can be separated into the gas components methane and carbon dioxide in a multi-stage process according to this embodiment. Alternatively, the various fluid reservoirs can be connected to the fluid discharge device 2 sequentially and manually by an operator.

According to FIG. 4, a fluid adsorption storage 12 filled with biogas, a secondary fluid storage 4, a primary fluid storage 3 and a pure fluid storage 5 are connected to the fluid discharge device 2 simultaneously. In a first rapid discharge phase, the fluid adsorption storage 12 is connected to a changeover valve 23 via a supply line 20, wherein the changeover valve connects the supply line 20 to an inlet valve 40 of the secondary fluid storage 4 in the switch position present in the first rapid discharge phase. In the first, rapid discharge phase, methane is primarily discharged from the fluid adsorption storage 12 into the secondary fluid storage unit 4, while carbon dioxide primarily remains attached to the adsorbents in the fluid adsorption storage 12. In a second, slow discharge phase, the fluid adsorption storage 12 is connected to the changeover valve 23 via the supply line 20, whereby the changeover valve, in the switch position present in the second, slow discharge phase, connects the supply line 20 to an inlet valve 30 of the primary fluid storage unit 3, which is not shown. In the second, slow discharge phase, carbon dioxide is primarily discharged from the fluid adsorption storage 12 into the primary fluid storage 3. The fluid adsorption storage 12 is essentially completely emptied at the end of the second, slow discharge phase.

In order to further fill the primary fluid storage 3 in particular, after completion of the second, slow discharge phase, another fluid adsorption storage 12 filled at the biogas production plant 9 is connected to the supply line 20 of the fluid discharge device. This full fluid adsorption storage 12 is partially emptied into the secondary fluid storage 4 in a third, fast discharge phase and into the primary fluid storage 3 in a subsequent fourth, slow discharge phase. By additionally introducing primarily carbon dioxide into the primary fluid storage 3, the pressure in the primary fluid storage 3 increases in this embodiment to essentially 6 MPa. As a result, despite the already high carbon dioxide content in the stored biogas, methane can be discharged primarily from the primary fluid storage 3 in a fifth, rapid discharge phase under the high pressure in the primary fluid storage 3. This methane can be discharged into the secondary fluid storage 4, for example. After completion of the fifth, rapid discharge phase, almost pure carbon dioxide can be discharged from the primary fluid storage 3 in a sixth, slow discharge phase. The carbon dioxide discharged in the sixth, slow discharge phase has, for example, a carbon dioxide concentration of essentially 99 vol. %. This essentially pure carbon dioxide is discharged from an outlet valve 39 of the primary fluid storage 3 via a two-way line 28, the discharge line 29 and the inlet valve 50 into the pure fluid storage 5.

According to FIG. 5, a fluid discharge device 2 has a supply line 20, a measuring unit 24, a mass flow controller 25, a suction pump 26, a control unit 27 and a changeover valve 23. The changeover valve 23 connects the outlet of the suction pump 26 to one of three discharge lines, namely the first discharge line 291, the second discharge line 292 or the third discharge line 293. The suction pump can, for example, be frequency-controlled or speed-controlled. The measuring unit 24 detects the pressure, temperature and gas concentration in the supply line 20 and generates corresponding measured values. The control unit 27 receives the measured values from the measuring unit 24. The mass flow controller 25 detects the mass flow or flow rate through the fluid discharge device 2 and generates corresponding measured values. The control unit 27 receives the measured values from the mass flow controller 25. Alternatively, the measuring unit 24 detects the pressure, temperature, gas concentration in the supply line 20 and also the mass flow or flow rate through the fluid discharge device 2 and generates corresponding measured values, whereby the control unit 27 also receives the measured values corresponding to the mass flow or flow rate from the measuring unit 24. The control unit 27 controls the operation of the mass flow controller 25 and the suction pump 26. For example, the control unit 27 activates the suction pump 26 when the measuring unit 24 detects a pressure below 2.5 MPa and the control unit 27 receives a control signal corresponding to this detected pressure. Preferably, the suction pump 26 has a variable compression ratio, i.e., a variable ratio between the outlet pressure and the inlet pressure of the suction pump 26. Furthermore, the suction pump 26 generates the level of suction pressure depending on the level of the detected pressure corresponding to the received control signal. For example, the suction pump 26 generates a suction pressure of 50 kPa when the measuring unit 24 detects a pressure below 2.5 MPa and above 250 kPa within the supply line 20 of the fluid discharge device 2, whereas the suction pump 26 generates a suction pressure of 500 Pa when the measuring unit 24 detects a pressure below 250 kPa within the supply line 20 of the fluid discharge device 2. The control unit 27 can be a regulating unit which, for example, controls the frequency or speed of the suction pump 26, in particular as a function of the measured values obtained by the measuring unit 24 or the mass flow controller 25. In other words, the measuring unit 24 and the mass flow controller 25 record the actual values, for example, of the pressure, temperature and gas concentration in the supply line 20 and the actual value of the mass flow or flow rate through the fluid discharge device 2 and forward these to the control unit 27 as a regulating unit. In the control unit 27 as the regulating unit, the actual values of the pressure, temperature, gas concentration and mass flow or flow rate are compared with corresponding target value values that are stored or at least retrievable in the control unit 27 as the regulating unit. If there is a deviation between the actual values and the target value values, a control algorithm in the control unit 27 as the regulating unit determines an adjustment of the operating parameters, for example of the suction pump 26 and the mass flow controller 25, and transmits correspondingly adjusted operating instructions to the suction pump 26 and the mass flow controller 25. The suction pump 26 is then operated, for example, at a changed frequency or at a changed speed. After the adjusted operating instructions have been transmitted, the mass flow controller 25 is operated, for example, in a changed position of a control valve, so that the size of the effective flow opening of the mass flow controller 25 after receiving the adjusted operating instructions is changed compared to the size of the effective flow opening before receiving the adjusted operating instructions.

The suction pump 26 can, for example, be operated in a passive mode as a first operating mode in such a way that it is inactive in this passive mode, i.e., it does not generate any suction pressure. In an active mode following the passive mode as a second operating mode, the suction pump 26 can, for example, be operated in such a way that it is active in the active mode and generates a suction pressure of approximately 3 MPa. Alternatively, the level of suction pressure generated in the active mode can follow a defined time curve. Alternatively, the level of suction pressure generated in active mode can follow a time curve that can be defined by an operator. The level of suction pressure generated in active mode can, for example, follow a time curve that essentially corresponds to a logarithmic or exponential relationship between the level of suction pressure and time. The level of suction pressure generated in active mode can, for example, also follow a time curve comprising a plurality of sections, each section being selectable in particular from a time curve that essentially corresponds to a linear, quadratic, logarithmic or exponential relationship between the level of suction pressure and time.

Alternatively, the suction pump 26 can be operated in the first operating mode in such a way that it is initially inactive, is then activated while still in the first operating mode, and then generates a low suction pressure in the first operating mode, for example at a level of 100 kPa. Alternatively, the suction pump 26 can be operated in the first operating mode in such a way that it is already active at the start of the first operating mode and generates a low suction pressure, for example 100 kPa. The level of suction pressure generated in the first operating mode can alternatively follow a defined time curve. Alternatively, the level of suction pressure generated in the first operating mode can follow a time curve that can be defined by an operator. The level of suction pressure generated in the first operating mode can, for example, follow a time curve that essentially corresponds to a logarithmic or exponential relationship between the level of suction pressure and time. The level of the suction pressure generated in the first operating mode can, for example, also follow a time curve comprising a plurality of sections, each section being selectable in particular from a time curve that essentially corresponds to a linear, quadratic, logarithmic or exponential relationship between the level of the suction pressure and time. Preferably, the maximum suction pressure generated by the suction pump 26 in the first operating mode is lower than the maximum suction pressure in the second operating mode. The changeover valve 23 may also be omitted and the fluid may be passed through the suction pump 26 in the first and second operating modes, which is controlled or regulated accordingly. For example, in passive mode, the suction pump may be passive or remain deactivated and allow the fluid to flow through, and only be activated in active mode.

Alternatively, the mass flow controller 25 can be arranged after the measuring unit 24 and before the suction pump 26.

The supply line 20 may comprise a first supply line, a second supply line and a third supply line, which can be fluidically connected to the measuring unit 24 by means of a supply line changeover valve. Switching between the first supply line, the second supply line and the third supply line is performed by means of the supply line changeover valve in the same way as switching between the discharge lines 291 to 293 by means of the changeover valve 23. By switching between the first supply line, the second supply line and the third supply line, several filled fluid reservoirs can be connected to the fluid discharge device 2 at the same time. For example, a fluid from a fluid reservoir connected to the first supply line can first be discharged through the fluid discharge device 2, then, after switching the supply line switch valve, a fluid from a fluid reservoir connected to the second supply line can be discharged through the fluid discharge device 2, and finally, after switching the supply line switch valve again, a fluid from a fluid reservoir connected to the third supply line can be discharged through the fluid discharge device 2.

According to FIG. 6, the fluid adsorption storage 1 is arranged on a tanker vehicle 7. Furthermore, a fluid discharge device 2 connected to the fluid adsorption storage 1 is arranged on the tanker vehicle 7. The fluid adsorption storage 1 of the tanker vehicle 7 can be filled at a biogas production plant 9 (not shown) and transport biogas, which comprises a gas mixture of methane and carbon dioxide, from the biogas production plant 9 (not shown) to a biomethane processing plant 8 (not shown). The system according to FIG. 6, comprising the tanker vehicle 7, the fluid adsorption storage 1 and the fluid discharge device 2, has the advantage that, after transport to the not shown biomethane processing plant 8, methane-rich biogas can first be removed from the system according to FIG. 6 in separate removal steps, followed by carbon dioxide-rich biogas. In other words, methane and carbon dioxide are already separated at the point of delivery of the biogas. This means that fewer or no further processing steps are required at the biomethane processing plant 8 (not shown) to separate methane and carbon dioxide.

List of reference signs

| | |
|---|---|
| 1 | fluid adsorption storage |
| 11 | fluid adsorption storage |
| 12 | fluid adsorption storage |
| 19 | outlet valve |
| 2 | fluid discharge device |
| 20 | supply line |
| 23 | changeover valve |
| 24 | measuring unit |
| 25 | mass flow controller |
| 26 | suction pump |
| 27 | control unit |
| 28 | two-way line |
| 29 | discharge line |
| 291 | first discharge line |
| 292 | second discharge line |
| 293 | third discharge line |
| 3 | primary fluid storage |
| 30 | inlet valve |
| 31 | primary fluid storage |
| 32 | primary fluid storage |
| 39 | outlet valve |
| 4 | secondary fluid storage |
| 40 | inlet valve |
| 41 | secondary fluid storage |
| 42 | secondary fluid storage |
| 5 | pure fluid storage |
| 50 | inlet valve |
| 7 | tanker vehicle |
| 8 | biomethane processing plant |
| 9 | biogas production plant |

1. Method, in particular for treating a fluid or fluid mixture, in particular a biogas mixture, comprising:
(a) providing a first fluid adsorption storage which
(a1) comprises at least one fluid with molecules of at least two different chemical substances, in particular a biogas mixture with methane molecules and carbon dioxide molecules, and
(a2) comprises at least one first adsorbent to which at least some of the molecules of the fluid are adsorbed as adsorbates, while other molecules are not adsorbed as adsorptives, and wherein the adsorptivities of the molecules of the different chemical substances are different, in particular the methane has a lower adsorptivity with the adsorbent, which preferably comprises activated carbon, than the carbon dioxide,
(a3) has at least one outlet valve and
(a4) is under a storage pressure from a high-pressure range, in particular between 2 and 10 MPa,
(b) providing a fluid discharge device comprising at least one supply line, at least one discharge line and at least one suction pump,
(c) wherein, in a first separation mode, the following process steps (d), (e) and (f) with the sub-steps (f1) to (f3) are performed on or with the first fluid adsorption storage:
(d) connecting the supply line of the fluid discharge device to the closed outlet valve of the first fluid adsorption storage;
(e) opening the outlet valve of the first fluid adsorption storage and allowing the fluid to flow out of the first fluid adsorption storage through the opened outlet valve, in a first operating mode, in particular in a passive mode without suction pressure from the suction pump, until the storage pressure reaches a low-pressure range, in particular below 2 MPa, and/or as long as the outflowing fluid predominantly contains molecules of the chemical substance with the lower adsorptivity,
(f) following the first operating mode, in particular following the passive mode, operating the fluid discharge device in a second operating mode, in particular in an active mode, with the following features:
(f1) generation of a suction pressure at the open outlet valve of the first fluid adsorption storage by means of the at least one suction pump;
(f2) desorbing or detaching adsorbates and/or molecules of the chemical substance with the higher adsorptivity that are still attached after the first operating mode, in particular after the passive mode, from the at least one adsorbent in the first fluid adsorption storage by means of the suction pressure of the suction pump, and
(f3) removing these desorbed or detached adsorbates and adsorptives from the first fluid adsorption storage by means of the fluid discharge device, whereby the removed fluid now predominantly contains molecules of the chemical substance with the higher adsorptivity.

2. Method according to claim 1, further comprising the following steps:
providing a primary fluid storage unit; and
connecting the discharge line of the fluid discharge device to at least one valve of the primary fluid storage;
wherein, in active mode, the fluid is fed through the discharge line of the fluid discharge device into the primary fluid storage.

3. Method according to claim 2, wherein the primary fluid storage device is a second fluid adsorption storage comprising at least one second adsorbent to which molecules of the fluid or adsorbates of the different chemical substances attach or are adsorbed, wherein, in particular, a second separation mode is performed with the second fluid adsorption storage, which has the corresponding features of the first separation mode according to feature (c) of claim 1 for the second fluid adsorption storage instead of the first fluid adsorption storage.

4. Method according to claim 1, further comprising:
providing a secondary fluid storage device; and
connecting the discharge line of the fluid discharge device to the secondary fluid storage;
wherein, in the first separation mode, the following process steps are further performed on or with the first fluid adsorption storage:
opening the outlet valve of the first fluid adsorption storage and allowing the fluid to flow out of the first fluid adsorption storage through the opened outlet valve in the first operating mode, in particular in the passive mode without suction pressure from the suction pump, until the storage pressure reaches a low-pressure range, in particular below 2 MPa, and/or as long as the outflowing fluid predominantly contains molecules of the chemical substance with the lower adsorptivity;
flowing of the fluid in the first operating mode, in particular in the passive mode, out of the first fluid adsorption storage through the discharge line of the fluid discharge device into the secondary fluid storage.

5. Method according to claim 3, wherein the fluid conducted in the second operating mode of the first separation mode, in particular in the active mode of the first separation mode, through the discharge line of the fluid discharge device into the second fluid adsorption storage, is stored in the second fluid adsorption storage at a storage pressure from a high-pressure range, in particular between 2 and 10 MPa, in particular using a compressor, the method, in particular, further comprising:
providing pure fluid storage;
connecting the discharge line of the fluid discharge device to the pure fluid storage;
wherein, in the second separation mode, the following process steps are further performed on or with the second fluid adsorption storage:
opening the outlet valve of the second fluid adsorption storage and allowing fluid to flow out of the second fluid adsorption storage through the opened outlet valve in a first operating mode of the second separation mode, in particular in a passive mode of the second separation mode without suction pressure from the suction pump, until the storage pressure in the second fluid adsorption storage reaches a low-pressure range, in particular below 2 MPa, and/or as long as the outflowing fluid predominantly contains molecules of the chemical substance with the lower adsorptivity,
following the first operating mode of the second separation mode, in particular following the passive mode of the second separation mode, operating the fluid discharge device in a second operating mode of the second separation mode, in particular in an active mode of the second separation mode, with the following features:
generating a suction pressure at the open outlet valve of the second fluid adsorption storage by means of the at least one suction pump;
desorbing or detaching adsorbates and/or molecules of the chemical substance with the higher adsorptivity that are still attached after the first operating mode of the second separation mode, in particular after the passive mode of the second separation mode, from the second adsorbent in the second fluid adsorption storage by means of the suction pressure of the suction pump, and
removing these desorbed or detached adsorbates and adsorptives from the second fluid adsorption storage via the fluid discharge device, whereby the removed fluid now predominantly contains molecules of the chemical substance with the higher adsorptivity
flowing the desorbed or detached adsorbates and adsorptives out of the second fluid adsorption storage through the discharge line of the fluid discharge device into the pure fluid storage.

6. Method according to claim 1, further comprising the following steps:
providing a mass flow controller, and
regulating the mass flow through the fluid discharge device to a mass flow target value, wherein the mass flow target value is in particular time-dependent or
wherein the level of the suction pressure is time-dependent and the suction pressure, in particular in the first operating mode and/or in the second operating mode, is generated by the suction pump along a predetermined or predeterminable time curve or
further comprising the provision of a control unit which controls the operation of the suction pump and/or the mass flow controller.

7. Method according to claim 1, further comprising the following steps:
providing a measuring unit for detecting measured values of one or more of the following measured variables:
pressure within the supply line of the fluid discharge device,
temperature within the supply line of the fluid discharge device,
mass flow through the fluid discharge device; and
fluid concentration within the supply line of the fluid discharge device; and
detecting measured values of the one or more measured variables by the measuring unit,
wherein, in particular, the control unit receives a control signal corresponding to the one or more detected measured values, and wherein the control unit activates the suction pump when the level of the detected pressure corresponding to the received control signal falls below a first threshold value,
wherein, in particular, the first threshold value is generally in a range between 250 kPa and 4 MPa, preferably in a range between 2 MPa and 3 MPa, and particularly preferably in a range between 2.3 MPa and 2.7 MPa, or wherein the first threshold value is generally in a range between two and a half times and forty times, preferably between twenty and thirty times, particularly preferably between twenty-three and twenty-seven times atmospheric pressure outside the first fluid adsorption storage,
wherein, in particular, the suction pump generates the suction pressure level as a function of the received control signal, in particular as a function of the detected pressure level corresponding to the received control signal and/or
wherein, in particular, the suction pump generates the level of suction pressure as a function of the level of the detected pressure corresponding to the received control signal, wherein the suction pump is operated in a first operating mode if the level of the detected pressure corresponding to the received control signal is above a second threshold value, and the suction pump is operated in a second operating mode if the level of the detected pressure corresponding to the received control signal is below the second threshold value, wherein the pressure generated in the second operating mode is greater than the pressure generated in the first operating mode, and wherein the second threshold value is generally between 100 kPa and 1.1 MPa, preferably in a range between 150 kPa and 750 kPa, and particularly preferably in a range between 200 kPa and 300 kPa, or wherein the second threshold value is generally in a range between one and eleven times, preferably between one and a half and seven and a half times, particularly preferably between two and three times atmospheric pressure outside the first fluid adsorption storage, wherein in particular the first operating mode corresponds to the first working mode and the second operating mode corresponds to the second working mode.

8. Method according to one claim 1, wherein the at least one fluid is a fluid mixture comprising a first fluid in a first fluid concentration and a second fluid in a second fluid concentration, wherein preferably the first fluid is carbon dioxide and the second fluid is methane,
wherein, in particular, the first fluid concentration in the fluid mixture flowing through the discharge line of the fluid discharge device in the second operating mode of the second separation mode, in particular in the active mode of the second separation mode, is greater than the first fluid concentration in the fluid mixture flowing through the discharge line of the fluid discharge device in the second operating mode of the first separation mode, in particular greater than in the active mode of the first separation mode,
wherein, optionally, the first fluid concentration in the fluid mixture flowing through the discharge line of the fluid discharge device in the second operating mode of the second separation mode, in particular in the active mode of the second separation mode, is essentially 99 vol. %.

9. Method according to claim 8, further comprising the provision of a control unit which controls the operation of the suction pump and/or the mass flow controller,
wherein the operation of the suction pump and/or the mass flow controller in the first separation mode is controlled by the control unit such that the first fluid concentration in the fluid mixture flowing through the discharge line of the fluid discharge device in the second operating mode of the first separation mode, in particular in the active mode of the first separation mode, is greater than the first fluid concentration in the fluid mixture stored in the fluid adsorption storage prior to the first separation mode,
wherein, in particular, the temporal temperature gradient and/or the temporal pressure gradient within the supply line of the fluid discharge device is regulated by means of the operation of the suction pump and/or the mass flow controller in the second operating mode of the first separation mode, in particular in the active mode of the first separation mode, such that in the second operating mode of the first separation mode, in particular in the active mode of the first separation mode, more adsorbate molecules of the first fluid are removed from the first adsorbent and pass into the fluid phase than adsorbate molecules of the second fluid.

10. Method according to claim 1, wherein the method for conditioning the first adsorbent is carried out before refilling the first fluid adsorption storage and wherein the removal of the one or more first adsorbates from the one or more first adsorbents of the first fluid adsorption storage increases the surface area of the one or more first adsorbents available for the adsorption of adsorbates.

11. Method according to claim 4, further comprising the following steps:
providing a primary fluid storage unit; and
connecting the discharge line of the fluid discharge device to at least one valve of the primary fluid storage;
wherein, in active mode, the fluid is fed through the discharge line of the fluid discharge device into the primary fluid storage,
further comprising the provision of a control unit which controls the operation of the suction pump and/or the mass flow controller,
wherein the fluid discharge device comprises a changeover valve which, in a first position, fluidically connects the outlet valve of the first fluid adsorption storage to the secondary fluid storage and, in a second position, fluidically connects the outlet valve of the first fluid adsorption storage fluidically to the primary fluid storage, and wherein the changeover valve is designed or configured to receive a changeover signal from the control unit for switching from the first position to the second position.

12. Method according to claim 11, further comprising the following steps:
providing a measuring unit for detecting measured values of one or more of the following measured variables:
pressure within the supply line of the fluid discharge device,
temperature within the supply line of the fluid discharge device,
mass flow through the fluid discharge device; and
fluid concentration within the supply line of the fluid discharge device; and
detecting measured values of the one or more measured variables by the measuring unit,
wherein the changeover signal is generated by the control unit as a function of the measured values of the one or more measured variables detected by the measuring unit.

13. Method according to claim 11, wherein the first operating mode of the first separation mode, in particular the passive mode of the first separation mode, the second operating mode of the first separation mode, in particular the active mode of the first separation mode, the first operating mode of the second separation mode, in particular the passive mode of the second separation mode, and the second operating mode of the second separation mode, in particular the active mode of the second separation mode, are controlled automatically by the control unit.

14. Method according to claim 8,
further comprising:
providing a secondary fluid storage device; and
connecting the discharge line of the fluid discharge device to the secondary fluid storage;
wherein, in the first separation mode, the following process steps are further performed on or with the first fluid adsorption storage:
opening the outlet valve of the first fluid adsorption storage and allowing the fluid to flow out of the first fluid adsorption storage through the opened outlet valve in the first operating mode, in particular in the passive mode without suction pressure from the suction pump, until the storage pressure reaches a low-pressure range, in particular below 2 MPa, and/or as long as the outflowing fluid predominantly contains molecules of the chemical substance with the lower adsorptivity;
flowing of the fluid in the first operating mode, in particular in the passive mode, out of the first fluid adsorption storage through the discharge line of the fluid discharge device into the secondary fluid storage,
further comprising the provision of a control unit which controls the operation of the suction pump and/or the mass flow controller,
wherein the operation of the suction pump and/or the mass flow controller in the first operating mode of the first separation mode, in particular in the passive mode of the first mode, is controlled by the control unit in such a way that the second fluid concentration in the fluid mixture flowing through the discharge line of the fluid discharge device in the first operating mode of the first separation mode, in particular in the passive mode of the first separation mode, is greater than the second fluid concentration in the fluid mixture stored in the first fluid adsorption storage prior to the first operating mode of the first separation mode, in particular prior to the passive mode of the first separation mode.

15. Method according to claim 14, wherein the temporal temperature gradient and/or the temporal pressure gradient within the supply line (20) of the fluid discharge device (2) is regulated by means of the operation of the suction pump (26) and/or the mass flow controller (25) in the first operating mode of the first separation mode, in particular in the passive mode of the first separation mode, such that in the first operating mode of the first separation mode, in particular in the passive mode of the first separation mode, more adsorbate molecules of the second fluid are removed from the first adsorbent and pass into the fluid phase than adsorbate molecules of the first fluid and/or
wherein the temporal pressure gradient within the supply line (20) of the fluid discharge device (2) in the first operating mode of the first separation mode, in particular in the passive mode of the first separation mode, is greater than in the second operating mode of the first separation mode, in particular greater than in the active mode of the first separation mode.

16. Fluid discharge device, in particular for use in a method according to claim 1, for a fluid or fluid mixture, in particular for a biogas mixture stored in a fluid adsorption storage,
wherein the device comprises a suction pump, and
wherein the device is designed or suitable for generating, by means of the suction pump, a suction pressure which draws one or more first adsorbates, in particular methane molecules, methanol molecules, hydrogen molecules or carbon dioxide molecules, adsorbed to at least one first adsorbent in a first fluid adsorption storage, and removes them in fluid phase via the fluid discharge from the first fluid adsorption storage.

17. Fluid discharge device according to claim 16,
wherein the level of the suction pressure is time-dependent and the suction pressure is generated by the suction pump along a predetermined or predeterminable time curve.

18. Fluid discharge device according to claim 16, wherein the device further comprises at least one of:
(i) a mass flow controller which is designed or configured to control the mass flow through the fluid discharge device to a mass flow target value, wherein the mass flow target value is in particular time-dependent,
(ii) a control unit which is designed or configured to control the operation of the suction pump and/or the mass flow controller,
(iii) a measuring unit for detecting measured values of one or more of the following measured variables:
pressure within the supply line of the fluid discharge device,
temperature within the supply line of the fluid discharge device,
mass flow through the fluid discharge device; and
fluid concentration within the supply line of the fluid discharge device.

19. System, comprising a fluid discharge device according to claim 16 and a first fluid adsorption storage comprising at least one first adsorbent, wherein the first adsorbent is preferably selected from a group comprising activated carbon, periodically mesoporous organosilicon dioxide, controllably porous glasses, mesoporous silicon dioxide, carbon-mesoporous Korea, monolithic carbon, in particular monolithic nanoporous carbon, and monomodal microporous carbon-based adsorbents.

20. System according to claim 19, having at least one of the following features:
(i) the first fluid adsorption storage comprises an outlet valve, and wherein, preferably, the first fluid adsorption storage can be fluidically connected to the supply line of the fluid discharge device via the outlet valve,
(ii) the system further comprises a primary fluid storage device, wherein the primary fluid storage device is preferably a second fluid adsorption storage comprising at least one second adsorbent, which is preferably selected from a group comprising activated carbon, periodically mesoporous organosilicon dioxide, controllably porous glasses, mesoporous silicon dioxide, carbon-mesoporous Korea, monolithic carbon, in particular monolithic nanoporous carbon, and monomodal microporous carbon-based adsorbents,
wherein, in particular, the primary fluid storage comprises an inlet valve, wherein the primary fluid storage is preferably fluidically connectable via the inlet valve 30) to the discharge line of the fluid discharge device
(iii) the first fluid adsorption storage is arranged in or on a motor vehicle or wherein the first fluid adsorption storage (is arranged in or on a trailer of a motor vehicle
(iv) wherein the fluid discharge device is arranged in or on a or the motor vehicle or wherein the fluid discharge device is arranged in or on a trailer of a or the motor vehicle,
(v) the primary fluid storage is arranged in or on a or the motor vehicle or wherein the primary fluid storage is arranged in or on a trailer of a or the motor vehicle (7),
(vi) the motor vehicle is a tanker vehicle.
